(19) United States
(12) Patent Application Publication
(10) Pub. No.: US 2008/0312241 A1
(43) Pub. Date: Dec. 18, 2008

(54) Cicletanine and PKC inhibitors in the treatment of pulmonary and cardiac disorders

(75) Inventors: Glenn V. Cornett, Palo Alto, CA (US); James Page, Laramie, WY (US); Wayne A. Jones, Palo Alto, CA (US); Karen Page, Laramie, WY (US)

Correspondence Address: GILEAD SCIENCES INC, 333 LAKESIDE DR, FOSTER CITY, CA, 94404, US

(73) Assignee: Gilead Sciences, Inc., Foster City, CA (US)

(21) Appl. No.: 12/006,825

(22) Filed: Jan. 3, 2008

Related U.S. Application Data
(60) Provisional application No. 60/883,338, filed on Jan. 3, 2007.

(51) Int. Cl. A61K 31/4985; A61K 31/4355; A61K 31/192; A61K 31/195; A61P 9/10; A61K 31/34; A61K 31/407; A61K 31/343

(52) U.S. Cl. 514/249; 514/302; 514/571; 514/410; 514/469; 514/470

(57) ABSTRACT

Embodiments of the present invention are related to novel therapeutic drugs, drug combinations, and associated methods for treating or preventing pulmonary disease, including pulmonary hypertension, pulmonary fibrosis, asthma and COPD, and heart failure, together with other pulmonary and cardiovascular diseases and their complications. More particularly, aspects of the present invention are related to the use of cicletanine and ruboxistaurin as monotherapies or in combination with other agents for treatment of disease. Cicletanine may be used as pure (+) or (−) enantiomers or as a racemic or non-racemic mixture of those enantiomers.

### CLAIM OF PRIORITY

The present application claims priority to U.S. Provisional Patent Application Ser. No. 60/883,338, filed Jan. 3, 2007, which is incorporated herein by reference.

### FIELD OF THE INVENTION

Embodiments of the present invention are related to using compositions of cicletanine and PKC inhibitors, either alone, or in combination with other agents, for the treatment of diseases such as pulmonary hypertension, chronic obstructive pulmonary disease (COPD), cor pulmonale, asthma, idiopathic pulmonary fibrosis, other pulmonary diseases and associated complications of these diseases.

### BACKGROUND OF THE INVENTION

### Pulmonary Hypertension

Pulmonary hypertension is a relatively rare disease in which, generally speaking, the pulmonary vasculature undergoes pathologic changes resulting in elevated pulmonary artery pressures with concomitant increase in right ventricular workload. The illness is typically progressive and fatal. Untreated survival is approximately 3 years. Treatment options are relatively limited

Current FDA approved treatments include prostacyclin analogs, endothelin receptor antagonists and phosphodiesterase inhibitors. These agents are approved only for primary, or class I pulmonary hypertension. Although these agents often produce some vasodilation, they are thought to exert their long term positive effects on the pulmonary vasculature through other means.

The cause of PH is not known and is likely multifactoral, rendering accurate prediction of efficacy for possible treatment approaches prior to clinical trials very difficult. Although several animal models have been developed, they are not reliably predictive of long term clinical benefit. The disease entity has been characterized by the World Health Organization (WHO) into several classes based upon pathologic states that may have some relevance to the underlying causes of the disease. It is generally thought that different treatment modalities may be required for the different classes, and indeed, some differences in response to treatment have been noted even within different sub categories of each individual class. So far, agents approved for treatment of WHO I category have not yet been approved for the other categories, and there have been several failed clinical trials for such agents in WHO category Ill. Even the approved drugs are often limited in application to certain functional levels within the class, based upon risk and benefit assessment. WHO category I is thought to have a prevalence of around 40,000 patients.

There is a critical need for further safe, effective and convenient pulmonary hypertension treatments. Not all patients respond to current therapies, and many continue a progressive downhill course even on treatment. The prostacyclin analogue agents are inconvenient: epoprostenol is continuously delivered from an ice chilled backpack via central venous catheter with attendant infection risks, and abrupt discontinuation results in dangerous rebound effects. Iloprost is inhaled from a nebulizer for 10 minutes 6-9 times daily. Treprostinil is delivered via continuous IV or subcutaneous infusion, but often causes severe injection site pain.

### Pulmonary Fibrosis

Pulmonary fibrosis is an even rarer condition than pulmonary hypertension. It can be caused by a variety of insults to the pulmonary parenchymal tissue, but most commonly there is no clear clause, hence it is referred to as idiopathic. This form of the disease has a US prevalence of around 10,000. It's pathophysiology is unclear, but results in loss of elastic tissue in the lung, resulting in restricted lung capacity and inadequate oxygenation. There are no fully satisfactory treatments, but some forms respond to immune suppression.

### Asthma and Chronic Obstructive Pulmonary Disease

These are inter-related chronic diseases characterized by increased reactivity of the small airways of the long, with increased mucous production and constriction. Chronic Obstructive Pulmonary Disease also involves the loss of alveolar tissue as well. They are a major cause of morbidity and mortality. Current treatments are largely palliative and do not slow the underlying disease progression. Current US prevalence is 19,000,000.

### Related Patents

The following U.S. patent publications and applications are hereby incorporated by reference in their entirety, including as they relate to the use of furopyridines, including cicletanine, and either alone or in combination with other agents, for the treatment of various diseases or physiologic dysfunctions and claim of priority is made also to each:

2006/0089374, published Apr. 27, 2006

11/035,231; 11/035,308; and 11/035,328 filed on Jan. 13, 2005

60/684,684 filed on May 26, 2005

60/612,323; and 60/612,369 filed on Sep. 22, 2004

### SUMMARY OF THE INVENTION

In accordance with the present invention, various embodiments of therapeutically beneficial formulations, including but not limited to oral formulations are disclosed, comprising a therapeutically effective amount of cicletanine alone, or in combination with one or more second agents, or of ruboxistaurin alone, or in combination with cicletanine and/or other agents for the treatment of pulmonary hypertension and other pulmonary diseases, as well as the complications arising from such diseases.

### Cicletanine

High dosage racemic cicletanine has been used somewhat successfully in clinical trials and in the practice of medicine as a hypertension agent. However, it has never been approved for treatment of disease other than as a thiazide like diuretic for treatment of essential hypertension in a few European countries. Its mechanism of action is poorly understood, but it is known in some instances to have diuretic effects and also to act as a vasodilator in isolated tissue models at very high doses, doses that are likely impossible to practically achieve in living organisms.

The inventors have unexpectedly discovered that Cicletanine is an effective agent for the treatment of pulmonary hypertension, despite the fact that it may act as a vasodilator, a class of drugs that has not been found to be generally effective for producing long term clinical improvement of pulmonary hypertension. The inventors also have identified means for further improving cicletanine effectiveness, and for targeting such improvements to specific classes of pulmonary hypertension. The inventors have unexpectedly discovered that Cicletanine will be effective for treatment of PH in all of the current WHO classes, based among other reasons upon its beneficial effects on endothelial function, which results in reduced tissue hyperproliferation and in improved coagulation parameters.

The inventors have unexpectedly found that cicletanine yields favorable, long-term hemodynamic and clinical improvements from baseline in pulmonary hypertension associated with left-sided heart failure.

The inventors have unexpectedly found that Cicletanine demonstrates clinical benefit in WHO class II patients as well. When dosed orally once daily at 50 mg daily, improvements in functional status as measured by improved 12 minute walking distance were observed.

The inventors have unexpectedly discovered that Cicletanine is effective in treatment of complications of pulmonary hypertension, such as heart failure. Heart failure (usually right-sided heart failure) is most common complication and the leading cause of death among pulmonary hypertension patients. We have found that Cicletanine is an effective agent for treatment of heart failure (WHO class III).

Natriuretic peptide level, a marker of heart failure is improved by oral administration of 150 mg of Cicletanine daily to a human subject with heart failure.

We have unexpectedly found good tolerance of Cicletanine in some patient populations at doses up to 400 mg. Consequently the favorable effects of cicletanine in heart failure can be achieved safely by increasing the dosage significantly above that used in current practice. This is particularly effective by use of means to reduce or modulate the magnitude of the kaliuretic, natriuretic and diuretic effects of Cicletanine. As part of aspects of the present invention, and as described below, we have also invented several means of accomplishing this objective.

### Endothelial Dysfunction and Nitric Oxide Production.

A number of chemical compounds produce elevations in vascular nitric oxide levels. Such elevation can be helpful in treatment of disease, but can also worsen some disease states. Many compounds that elevate nitric oxide quickly lose efficacy over a matter of a few hours or days (tachyphylaxis) and require episodic dosing. During the off period of such dosing the disease process may continue or worsen. We have found that cicletanine increases nitric oxide in deficiency states without producing excessive levels that actually cause tissue damage. It also avoids or prevents tachyphylaxis. We have discovered that a compound, which we call a nitric oxide modulating agent is effective in treating numerous cardiac and pulmonary disorders, by improving the function of vascular endothelium. According to aspects of our present invention, furopyridine compounds and cicletanine in particular have the desired nitric oxide modulating properties.

We have found that Cicletanine produces increased levels of nitric oxide in vascular tissue and that this effect is persistent across a wide range of concentrations, corresponding to human doses ranging from 1 mg daily to several thousand milligrams daily. We have found that it does so by enhancing endogenous nitric oxide production and it also avoids tachyphylaxis to its own effects and prevents tachyphylaxis to other nitrogen enhancing drugs.

Accordingly we have found that this effect provides and allows effective treatment of all classes of pulmonary hypertension and heart failure as well as other cardiac and pulmonary pathologies. We have further found that this effect is more prominent in (−) cicletanine than in (+) cicletanine.

### Ruboxistaurin

Ruboxistaurin is a protein kinase C beta isoform inhibitor. It has undergone extensive investigation for treatment of diabetes complications with equivocal results. The FDA has recently notified its sponsor that a large additional successful clinical trial will be required before the drug can be approved in the US for treatment of Diabetic complications.

We have unexpectedly found that the characteristics of ruboxistaurin make it suitable agent for treatment of pulmonary hypertension, alone or in combination with Cicletanine. In particular it modulates the adverse effects of vascular endothelial dysfunction and dysregulated nitric oxide production. We propose that inhibition of the beta isoform of protein kinase C will have favorable effects upon nitric oxide levels.

We have unexpectedly found that ruboxistaurin, with its specific inhibition of PKC-beta, will have favorable effects, either alone or in combination with cicletanine, on pulmonary hypertension as well as on other cardiac and pulmonary disorders.

### DETAILED DESCRIPTION OF THE INVENTION

Aspects of some embodiments of the present invention include, inter alia, the use of nitric oxide modulating agents as follows:

(1) In particular furopyridines and more in particular cicletanine in the treatment of pulmonary and cardiac disease.

(2) In particular PKC-beta inhibitors and more in particular ruboxistaurin and cicletanine (either alone or in combination with each other) in the treatment of pulmonary and cardiac disease.

(3) Enhancement of the therapeutic efficacy of nitric oxide modulating agents, in particular furopyridines, more in particular Cicletanine, through manipulation of delivery, metabolism, cofactor availability, nitrogen and other substrate availability, and enantiomeric ratio selection.

“Cicletanine” when used herein when unmodified by such descriptors as racemic, enantiomeric, non-racemic, is intended to include any and all of these chemical entities: racemic cicletanine, (−) cicletanine enantiomer, (+) cicletanine enantiomer, non-racemic mixtures of the two cicletanine enantiomers.

A successful clinical trial of one of the embodiments comprised use of racemic cicletanine with the dosage initiated at 50 mg daily and titrated as tolerated to 150 mg daily in a patient with Class I Pulmonary Hypertension. This treatment has resulted in substantial improvement in functional status as measured by a more than 12 fold increase in walking distance and substantial reductions in natriuretic peptide serum levels, demonstrating improved cardiac function.

Another embodiment comprises the use of 50 mg oral daily doses of racemic cicletanine for treatment of Class II Pulmonary Hypertension, resulting in improved walking distance values.

Yet another embodiment comprises the use of 50 mg oral daily doses of racemic cicletanine for treatment of Class III Pulmonary Hypertension, resulting in improved NYHA functional status.

We have determined that there are substantial differences between individuals, as much as 2 orders of magnitude or greater, in metabolism of cicletanine enantiomers. Consequently, the certain embodiments of the invention will include a wide range of doses, titrated to main effects and side effects. Differences in absorption, distribution, and excretion may further increase the dose range.

Use of drug blood levels, biomarker levels, and clinical response allow particularization of individual doses to maximize effect and minimize adverse side effects.

To account for variations in absorption, distribution, metabolism and excretion, as well as differences in pathophysiology of the different disease classes certain embodiments of the invention include doses as low as 1 mg, and as high as 10,000 milligrams of cicletanine. Use of drug blood levels, biomarker levels, and clinical response allow particularization of individual doses to maximize effect and minimize adverse side effects.

In some embodiments of the invention, the various cicletanine and/or ruboxistaurin compositions may be the only active ingredients of the formulation. In other embodiments, a second, third, or fourth agent may be combined along with the cicletanine and/or ruboxistaurin. “Agent”, as used herein, refers to any pharmaceutically active agent other than cicletanine or ruboxistaurin. Such agents may be by themselves effective agents for treatment of pulmonary disease, or may enhance the effectiveness of cicletanine, ruboxistaurin or other agents contained in the combination therapy. Such agents may include prostacyclins and their analogues, xprostacyclin inducers, endothelin antagonists, phosphodiesterase inhibitors, anti-histamines, calcium channel blockers, cGMP activators, nitrogen or nitric oxide enhancers or donors for treatment of pulmonary hypertension. For heart failure such agents may include beta blockers, digitalis derivatives, phosphodiesterase inhibitors, vasodilators, and diuretics. For asthma, COPD, and pulmonary fibrosis, such agents include immune modulators such as steroids, leukotriene inhibitors, beta agonists, and mast cell inhibitors such as cromolyn. In many cases the range of diseases and associated complications or sequalae that receive benefit from cicletanine or ruboxistaurin alone are understood to derive further benefit from the combination formulations.

In accordance of the other embodiments of the present invention, an oral therapeutic formulation is disclosed wherein sufficient cicletanine is contained to improve pulmonary hemodynamics without excessive lowering of systemic blood pressure. Excessive reduction of systemic blood pressure has been a limiting side effect in many agents potentially useful for treatment of pulmonary hypertension. Our data shows that doses up to 150 mg have minimal effects on systemic blood pressure in normotensive patients. Use of appropriate delivery or other mechanisms to optimize blood levels of individual isomers will allow even higher dosing without adverse impacts on systemic blood pressures.

Various alternate embodiments may include the use, individually or in combination, of preparations of:
(1) Cicletanine where the (−) isomer is emphasized (e.g., in a non-racemic mixture of the two isomers that favors the (−) isomer) or used in its pure form.
(2) Ruboxistaurin
(3) A combination of cicletanine (pure (−)cicletanine or a mixture of isomers favoring (−)cicletanine) and ruboxistaurin.
(4) Racemic Cicletanine alone or in combination with ruboxistaurin
(5) Cicletanine alone or in combination with ruboxistaurin, with enhancement of therapeutic efficacy via modulation of eNOS cofactor availability, absorption or metabolism.

Another embodiment may include the treatment of patients having pulmonary hypertension and other pulmonary or cardiac diseases with, either singly or in combination:
(1) Cicletanine where the (−) isomer is emphasized (e.g., in a non-racemic mixture of the two isomers that favors the (−) isomer) or used in its pure form.
(2) Ruboxistaurin
(3) A combination of cicletanine (pure (−)cicletanine or a mixture of isomers favoring (−)cicletanine) and ruboxistaurin.

Another of the embodiments specifically involves the use of ruboxistaurin and/or cicletanine in the treatment of WHO group I patients, [including a strategy wherein (−) cicletanine isomer is emphasized (e.g., in a non-racemic mixture of the two isomers that favors the (−) isomer) or used in its pure form], in the treatment of patients with pulmonary hypertension of WHO Group I. With the demonstration of (−) cicletanine as a nitric oxide enhancing agent, the endothelial dysfunction (i.e., aberrant nitric oxide regulation) driving the pathology of this type of pulmonary hypertension (e.g., idiopathic or associated with connective tissue disease) is addressed in a clinically-favorable fashion.

An embodiment in treatment of class I pulmonary hypertension may include initiation of therapy at 50 mg oral daily dosing with titration upward as tolerated. This embodiment may be enhanced by use of serum drug and biomarker measurements to adjust dosing.

Another of the embodiments specifically involves the use of ruboxistaurin and/or cicletanine [including a strategy wherein (−)cicletanine isomer is emphasized (e.g., in a non-racemic mixture of the two isomers that favors the (−) isomer) or used in its pure form] in the treatment of patients with pulmonary hypertension of WHO Group II. With the demonstration of (−)cicletanine as a superior nitric oxide enhancing agent, the endothelial dysfunction (i.e., nitric oxide regulation) driving the pathology of this type of pulmonary hypertension (e.g., PH associated with hypoxia, such as that seen in chronic obstructive pulmonary disease) is addressed in a clinically-favorable fashion. Further, in certain embodiments, emphasizing the (−) enantiomer or using it in its pure form yields further clinical benefits over those derived from racemic Cicletanine.

An embodiment in treatment of class II pulmonary hypertension may comprise initiation of therapy at 50 mg oral daily dosing with titration upward as tolerated. This embodiment may be enhanced by use of serum drug and biomarker measurements to adjust dosing.

Another of the embodiments may comprise the use of ruboxistaurin and/or cicletanine [including a strategy wherein the (−)cicletanine isomer is emphasized (e.g., in a non-racemic mixture of the two isomers that favors the (−) isomer) or used in its pure form] in the treatment of patients with pulmonary hypertension of WHO Group III. With the demonstration of (−)cicletanine as a nitric oxide enhancing agent, the endothelial dysfunction (i.e., aberrant nitric oxide regulation) driving the pathology of this type of pulmonary hypertension (e.g., PH associated with left-sided heart failure) is addressed in a clinically-favorable fashion. Further, in some embodiments, emphasizing the (−) enantiomer or using it in its pure form will yield further clinical benefits.

An embodiment in treatment of class III pulmonary hypertension may comprise initiation of therapy at 50 mg oral daily dosing with titration upward as tolerated. This embodiment may be enhanced by use of serum drug and biomarker measurements to adjust dosing.

Another embodiment may comprise the use of ruboxistaurin and/or cicletanine [including a strategy wherein the (−)cicletanine isomer is emphasized (e.g., in a non-racemic mixture of the two isomers that favors the (−) isomer) or used in its pure form] in the treatment of patients with pulmonary hypertension of WHO Group IV. The ability to address the endothelial dysfunction underlying some of the pathology of this disease with an agent distinctively able to address nitric oxide levels provides meaningful clinical benefits to patients with this disease.

An embodiment in treatment of class IV pulmonary hypertension may comprise initiation of therapy at 50 mg oral daily dosing with titration upward as tolerated. This embodiment may be enhanced by use of serum drug and biomarker measurements to adjust dosing.

Another embodiment may comprise the use of ruboxistaurin and/or cicletanine [including a strategy wherein the (−)cicletanine isomer is emphasized (e.g., in a non-racemic mixture of the two isomers that favors the (−) isomer) or used in its pure form] in the treatment of patients with pulmonary hypertension of WHO Group V. The ability to address the underlying nitric oxide dysregulation (expected to result from hypoxia, shear stress, etc. associated with the disease) in these patients achieves favorable clinical improvements.

An embodiment in treatment of class IV pulmonary hypertension may comprise initiation of therapy at 50 mg oral daily dosing with titration upward as tolerated. This embodiment may be enhanced by use of serum drug and biomarker measurements to adjust dosing.

As previously discussed, in some embodiments when treating heart-failure patients with higher doses of cicletanine, the tendency of the diuretic, natriuretic and particularly the hypokalemic effects of the drug to increase significantly as the dosage reaches and exceeds 150 mg per day is a factor limiting therapeutic effectiveness. It is proposed that these diuretic, natriuretic and hypokalemic effects of cicletanine are due predominantly or overwhelmingly to the drug's (+) isomer. Heart failure patients, given the typical nature of their pharmaceutical polytherapy (typically involving a cardiac glycoside, a loop diuretic, a potassium-sparing diuretic, etc.), can be exquisitely sensitive to changes in potassium balance. Use in these patients of a cicletanine preparation that predominates in the (−) isomer or uses only the (−) isomer will therefore, in some embodiments, optimize the use of cicletanine in heart-failure and pulmonary hypertension patients.

We have identified other means of minimizing diuretic effects as well. These include, in some embodiments, transdermal, transmucosal, inhaled, and depot parenteral administration of Cicletanine in order to reduce first pass hepatic metabolism, which produces a Cicletanine metabolite that is responsible for diuretic activity, and converts the free Cicletanine responsible for its other therapeutic effects, into a metabolite that is clinically inactive.

Use of a time release formulation, in some embodiments, enhances Cicletanine effectiveness in cardiac disease and pulmonary hypertension, even though Cicletanine's biological activity is long lasting. An extended release formulation reduces peak drug levels associated with natriuresis, kaliuresis, and diuresis, while still achieving adequate levels for its endothelial protective activities. In some instances there are differences in the clearance rate between the active and inactive forms of Cicletanine that will make this strategy even more useful.

According to some embodiments, use of agents acting in the liver to inhibit or enhance certain metabolic pathways allow for a more effective ratio of active and less active forms of Cicletanine. Specifically, certain members of the fibrate class of drugs act to inhibit glucoronidation, which converts Cicletanine into an inactive metabolite. Other metabolic enhancers/inhibitors may also be used.

In an embodiment, a formulation incorporating 600 mg of gemfibrozil and a therapeutic amount of cicletanine as elsewhere discussed are administered once daily by mouth. Titration with initial amounts of cicletanine beginning at 10 mg and increasing to as much as 10,000 mg. are performed using clinical effect, serum levels of active drug or other biomarkers.

According to some embodiments of the present invention, we also use nitrate, nitrite, amine, and amide salts and esters or other chemically bound nitrogen containing combinations with Cicletanine and its enantiomers to further enhance the beneficial effects of Cicletanine on the endothelium by providing further enhancement in nitric oxide levels.

In some embodiments, we use a combination formulation of cicletanine or its enantiomers or combinations of its enantiomers and a second agent that provides additional nitrogen availability, such as nitroglycerin, isosorbide dinitrate, arginine or arginine containing compounds.

Another enhancement of some embodiments is the use of cofactors and or substrates for nitric oxide production in combination with Cicletanine.

Another embodiment may comprise use of a therapeutic amount of cicletanine formulated with a therapeutic amount of tetrahydrobiopterin (BH4) and or Arginine. The dose of cicletanine is titrated as heretofore discussed. Arginine dose is titrated from 200 mg to 1000 mg daily. BH4 or an orally administered analogue thereof is incorporated in a therapeutically active amount.

Another enhancement may comprise use of nitrogen providing chemical entities that are chemically bound to the cicletanine molecule to enhance its effectiveness in modulating endothelial function.

The embodiments disclosed herein may comprise and/or be applied to racemic, non-racemic and pure enantiomeric forms of Cicletanine.

Another embodiment may comprise use of the other enhancement strategies noted above applied to one or more of the following:

racemic cicletanine

pure (−) cicletanine

pure (+) cicletanine

a non-racemic mixture of (−) and (+) cicletanine

In some embodiments, pure(+) cicletanine has beneficial effects across a narrower dose range than that of (−) cicletanine and racemic cicletanine, wherein enhanced prostacyclin synthesis is mediated. This effect can be maximized in some embodiments by dose adjustment to achieve active drug levels in the range of 10-9 to 10-7 moles/liter. Use of prostacyclin/thromboxane ratios and prostacyclin metabolite assays, as well as serum drug levels allows targeting for maximum benefit with minimum adverse side effects.

An embodiment of (+) cicletanine is upward titration of once daily oral dosing beginning at 10 mg and titrating upward to 200 mg. This embodiment in some instances is particularly appropriate for patients in whom the diuretic and natriuretic effects of (+) cicletanine are well tolerated or helpful.

Another embodiment of the invention is use of cicletanine alone or in combination with other agents for the treatment of asthma and COPD via cicletanine's antihistaminergic and notric oxide enhancing properties.

In yet another embodiment, cicletanine is used to reduce the side effects of other medications, such as injection site inflammation from prostacyclins or peripheral edema from calcium channel blockers.

In still another embodiment, cicletanine is used to enhance the effect of other therapeutic agents, thereby allowing lower dosing with an enhanced side effect profile.

In still another embodiment, cicletanine is used in combination with another agent or agents to allow a combined medication dosage to be more effective than that of similar doses of the individual agents, thereby reducing side effect frequency or severity.

In embodiments of the invention, the combination therapies comprise fixed does (of each component), in single tablet or capsule form, or other appropriate oral formulation with appropriate excipients, dissolution enhancers and stabilizers. Among other advantages, combination therapy simplifies treatment regimens, and supports patient compliance. Provision of multiple combination does still allows for variation in total doses for appropriate tailoring of therapy

### Human Clinical Studies

Certain aspects of the present invention are embodied and illustrated in the following examples. While each of the compositions below involve total dosages of 150 mg, therapeutic dosages in related Examples may range from 1 mg to 10000 mg. Additionally, the medications described may include modifications to enhance effect as discussed above, as well as combinations with one or more of any members of the classes of drugs including prostacyclin analogues such as eprostenol, iloprost, and treprostinil, endothelin antagonists, such as bosentan, PDE inhibitors, such as sildenafil, calcium channel blockers, such as nifedipine, amlodipine, and nicardipine, nitrogen donors, such as sodium nitroprusside, and cGMP inducers or enhancers, such as atrial natriuretic peptide, and serotonin inhibitors.

### Human Study Examples

### EXAMPLE 1

A 33 year old female patient with idiopathic pulmonary arterial hypertension, despite having been for some time on high doses of all three classes of approved pulmonary hypertension drugs (endothelin receptor antagonist, prostacyclin, and PDE5 inhibitor), was in heart failure and deteriorating rapidly. The patient had finally been stabilized in the intensive care unit with continuously inhaled nitric oxide. Cicletanine therapy was inititiated—oral once daily, titrating from 50 mg to 150 mg/day over three days—day 1 at 50 mg, day 2 at 100 mg, day 3 and thereafter at 150 mg. Within three days from initiation of cicletanine therapy, the patient was weaned off nitric oxide and released from the intensive care unit; she was home within 5 days of initiation of cicletanine treatment. The patient has been on the cicletanine therapy for four and a half months.

The patient has manifested a significant improvement in 6-minute walking distance, which was less than 30 meters immediately prior to cicletanine initiation, and was over 200 meters after one month of cicletanine therapy and was over 400 meters from the second month of cicletanine therapy onward.

Also, levels of N-terminal pro-B-type natriuretic peptide (an accepted biomarker of cardiac failure) for the patient have fallen significantly. Immediately prior to cicletanine therapy the marker was at 4409; within a few days of cicletanine therapy biomarker levels had fallen to less than 2000, and have stayed near 2000 for approximately four months.

Finally, quality of life of the patient has improved significantly. The patient, formerly bedridden, is now able to perform housework and take regular exercise.

### EXAMPLE 2

A cicletanine formulation comprising 200 mg of (+) cicletanine is administered via an oral route once daily, to patients with WHO group I pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 2a

A cicletanine formulation comprising 180 mg of (+) cicletanine and 20 mg of (−) cicletanine is administered via an oral route once daily, to patients with WHO group I pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 3

A cicletanine formulation comprising 160 mg of (+) cicletanine and 40 mg of (−) cicletanine is administered via an oral route once daily, to patients with WHO group I pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 4

A cicletanine formulation comprising 140 mg of (+) cicletanine and 60 mg of (−) cicletanine is administered via an oral route once daily, to patients with WHO group I pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing midrange fluid overload.

### EXAMPLE 5

A cicletanine formulation comprising 120 mg of (+) cicletanine and 80 mg of (−) cicletanine is administered via an oral route once daily, to patients with WHO group I pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 6

A cicletanine formulation comprising 100 mg of (+) cicletanine and 100 mg of (−) cicletanine is administered via an oral route once daily, to patients with WHO group I pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mid range diuretic effect and is most appropriate for patients experiencing intermediate fluid overload.

### EXAMPLE 7

A cicletanine formulation comprising 80 mg of (+) cicletanine and 120 mg of (−) cicletanine is administered via an oral route once daily, to patients with WHO group I pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 8

A cicletanine formulation comprising 60 mg of (+) cicletanine and 140 mg of (−) cicletanine is administered via an oral route once daily, to patients with WHO group I pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 9

A cicletanine formulation comprising 40 mg of (+) cicletanine and 160 mg of (−) cicletanine is administered via an oral route once daily, to patients with WHO group I pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effect and is most appropriate for patients experiencing modest fluid overload.

### EXAMPLE 10

A cicletanine formulation comprising 20 mg of (+) cicletanine and 180 mg of (−) cicletanine is administered via an oral route once daily, to patients with WHO group I pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effects and is most appropriate for patients experiencing modest fluid overload or no fluid overload.

### EXAMPLE 11

A cicletanine formulation comprising 200 mg of (−) cicletanine is administered via an oral route once daily, to patients with WHO group I pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

This formulation has little or no diuretic effect, and maximal nitric oxide effect and is best suited to patients with no fluid overload.

### EXAMPLE 12

A cicletanine formulation comprising 200 mg of (+) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 13

A cicletanine formulation comprising 180 mg of (+) cicletanine and 20 mg of (−) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effects and is most appropriate for patients experiencing modest fluid overload or no fluid overload.

### EXAMPLE 14

A cicletanine formulation comprising 160 mg of (+) cicletanine and 40 mg of (−) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 15

A cicletanine formulation comprising 140 mg of (+) cicletanine and 60 mg of (−) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing midrange fluid overload.

### EXAMPLE 16

A cicletanine formulation comprising 120 mg of (+) cicletanine and 80 mg of (−) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 17

A cicletanine formulation comprising 100 mg of (+) cicletanine and 100 mg of (−) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mid range diuretic effect and is most appropriate for patients experiencing intermediate fluid overload.

### EXAMPLE 18

A cicletanine formulation comprising 80 mg of (+) cicletanine and 120 mg of (−) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 19

A cicletanine formulation comprising 60 mg of (+) cicletanine and 140 mg of (−) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 20

A cicletanine formulation comprising 40 mg of (+) cicletanine and 160 mg of (−) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effect and is most appropriate for patients experiencing modest fluid overload.

### EXAMPLE 21

A cicletanine formulation comprising 20 mg of (+) cicletanine and 180 mg of (−) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effects and is most appropriate for patients experiencing modest fluid overload or no fluid overload.

### EXAMPLE 22

A cicletanine formulation comprising 200 mg of (−) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

This formulation has little or no diuretic effect, and maximal nitric oxide effect and is best suited to patients with no fluid overload.

### EXAMPLE 23

A cicletanine formulation comprising 200 mg of (+) cicletanine is administered via an oral route once daily, to patients with WHO group II pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effects and is most appropriate for patients experiencing modest fluid overload or no fluid overload.

### EXAMPLE 24

A cicletanine formulation comprising 180 mg of (+) cicletanine and 20 mg of (−) cicletanine is administered via an oral route once daily, to patients with WHO group III pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 25

A cicletanine formulation comprising 160 mg of (+) cicletanine and 40 mg of (−) cicletanine is administered via an oral route once daily, to patients with WHO group III pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 26

A cicletanine formulation comprising 140 mg of (+) cicletanine and 60 mg of (−) cicletanine is administered via an oral route once daily, to patients with WHO group III pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing midrange fluid overload.

### EXAMPLE 27

A cicletanine formulation comprising 120 mg of (+) cicletanine and 80 mg of (−) cicletanine is administered via an oral route once daily, to patients with WHO group III pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 28

A cicletanine formulation comprising 100 mg of (+) cicletanine and 100 mg of (−) cicletanine is administered via an oral route once daily, to patients with WHO group III pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mid range diuretic effect and is most appropriate for patients experiencing intermediate fluid overload.

### EXAMPLE 29

A cicletanine formulation comprising 80 mg of (+) cicletanine and 120 mg of (−) cicletanine is administered via an oral route once daily, to patients with WHO group III pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 30

A cicletanine formulation comprising 60 mg of (+) cicletanine and 140 mg of (−) cicletanine is administered via an oral route once daily, to patients with WHO group III pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 31

A cicletanine formulation comprising 40 mg of (+) cicletanine and 160 mg of (−) cicletanine is administered via an oral route once daily, to patients with WHO group III pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effect and is most appropriate for patients experiencing modest fluid overload.

### EXAMPLE 32

A cicletanine formulation comprising 20 mg of (+) cicletanine and 180 mg of (−) cicletanine is administered via an oral route once daily, to patients with WHO group III pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effects and is most appropriate for patients experiencing modest fluid overload or no fluid overload.

### EXAMPLE 33

A cicletanine formulation comprising 200 mg of (−) cicletanine is administered via an oral route once daily, to patients with WHO group III pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effects and is most appropriate for patients experiencing modest fluid overload or no fluid overload.

### EXAMPLE 34

A cicletanine formulation comprising 200 mg of (+) cicletanine is administered via an oral route once daily, to patients with WHO group IV pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 35

A cicletanine formulation comprising 180 mg of (+) cicletanine and 20 mg of (−) cicletanine is administered via an oral route once daily, to patients with WHO group IV pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 36

A cicletanine formulation comprising 160 mg of (+) cicletanine and 40 mg of (−) cicletanine is administered via an oral route once daily, to patients with WHO group IV pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 37

A cicletanine formulation comprising 140 mg of (+) cicletanine and 60 mg of (−) cicletanine is administered via an oral route once daily, to patients with WHO group IV pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing midrange fluid overload.

### EXAMPLE 38

A cicletanine formulation comprising 120 mg of (+) cicletanine and 80 mg of (−) cicletanine is administered via an oral route once daily, to patients with WHO group IV pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 39

A cicletanine formulation comprising 100 mg of (+) cicletanine and 100 mg of (−) cicletanine is administered via an oral route once daily, to patients with WHO group IV pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mid range diuretic effect and is most appropriate for patients experiencing intermediate fluid overload.

### EXAMPLE 40

A cicletanine formulation comprising 80 mg of (+) cicletanine and 120 mg of (−) cicletanine is administered via an oral route once daily, to patients with WHO group IV pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 41

A cicletanine formulation comprising 60 mg of (+) cicletanine and 140 mg of (−) cicletanine is administered via an oral route once daily, to patients with WHO group IV pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 42

A cicletanine formulation comprising 40 mg of (+) cicletanine and 160 mg of (−) cicletanine is administered via an oral route once daily, to patients with WHO group IV pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effect and is most appropriate for patients experiencing modest fluid overload.

### EXAMPLE 43

A cicletanine formulation comprising 20 mg of (+) cicletanine and 180 mg of (−) cicletanine is administered via an oral route once daily, to patients with WHO group IV pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effects and is most appropriate for patients experiencing modest fluid overload or no fluid overload.

### EXAMPLE 44

A cicletanine formulation comprising 200 mg of (−) cicletanine is administered via an oral route once daily, to patients with WHO group IV pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

This formulation has little or no diuretic effect, and maximal nitric oxide effect and is best suited to patients with no fluid overload.

### EXAMPLE 45

A cicletanine formulation comprising 200 mg of (+) cicletanine is administered via an oral route once daily, to patients with WHO group V pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 46

A cicletanine formulation comprising 180 mg of (+) cicletanine and 20 mg of (−) cicletanine is administered via an oral route once daily, to patients with WHO group V pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 47

A cicletanine formulation comprising 160 mg of (+) cicletanine and 40 mg of (−) cicletanine is administered via an oral route once daily, to patients with WHO group V pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 48

A cicletanine formulation comprising 140 mg of (+) cicletanine and 60 mg of (−) cicletanine is administered via an oral route once daily, to patients with WHO group V pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing midrange fluid overload.

### EXAMPLE 49

A cicletanine formulation comprising 120 mg of (+) cicletanine and 80 mg of (−) cicletanine is administered via an oral route once daily, to patients with WHO group V pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 50

A cicletanine formulation comprising 100 mg of (+) cicletanine and 100 mg of (−) cicletanine is administered via an oral route once daily, to patients with WHO group V pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mid range diuretic effect and is most appropriate for patients experiencing intermediate fluid overload.

### EXAMPLE 51

A cicletanine formulation comprising 80 mg of (+) cicletanine and 120 mg of (−) cicletanine is administered via an oral route once daily, to patients with WHO group V pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 52

A cicletanine formulation comprising 60 mg of (+) cicletanine and 140 mg of (−) cicletanine is administered via an oral route once daily, to patients with WHO group V pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 53

A cicletanine formulation comprising 40 mg of (+) cicletanine and 160 mg of (−) cicletanine is administered via an oral route once daily, to patients with WHO group V pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effect and is most appropriate for patients experiencing modest fluid overload.

### EXAMPLE 54

A cicletanine formulation comprising 20 mg of (+) cicletanine and 180 mg of (−) cicletanine is administered via an oral route once daily, to patients with WHO group V pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effects and is most appropriate for patients experiencing modest fluid overload or no fluid overload.

### EXAMPLE 55

A cicletanine formulation comprising 200 mg of (−) cicletanine is administered via an oral route once daily, to patients with WHO group V pulmonary hypertension. Cicletanine is administered alone or in combination with other classes of drugs as discussed above.

As a consequence pulmonary hemodynamics are improved, systemic blood pressure remains within acceptable limits, and in addition one or more of the following parameters is improved: BNP levels, walking distance, prostacyclin/thromboxane ratio, and NYHA functional class score. Metabolic parameters remain unchanged or improved. Such improvements remain above baseline for at least 3 months.

This formulation has little or no diuretic effect, and maximal nitric oxide effect, and is best suited to patients with no fluid overload.

### EXAMPLE 56

A cicletanine formulation comprising 200 mg of (+) cicletanine is administered via an oral route once daily, to patients with asthma, COPD, and or pulmonary fibrosis. Cicletanine is administered alone or in combination with other classes of drugs as discussed above.

As a consequence pulmonary function is improved or stabilized, systemic blood pressure remains within acceptable limits, and in addition one or more of the following parameters is improved: FEV1, O2 saturation, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 57

A cicletanine formulation comprising 180 mg of (+) cicletanine and 20 mg of (−) cicletanine is administered via an oral route once daily, to patients with asthma or COPD or pulmonary fibrosis. Cicletanine is administered alone or in combination with other classes of drugs as discussed above.

As a consequence pulmonary function is improved, systemic blood pressure remains within acceptable limits, and in addition one or more of the following parameters is improved: FEV1, O2 saturation, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 58

A cicletanine formulation comprising 160 mg of (+) cicletanine and 40 mg of (−) cicletanine is administered via an oral route once daily, to patients with asthma or COPD or pulmonary fibrosis. Cicletanine is administered alone or in combination with other classes of drugs as discussed above.

As a consequence pulmonary function is improved, systemic blood pressure remains within acceptable limits, and in addition one or more of the following parameters is improved: FEV1, O2 saturation, walking distance, prostacyclin/thromboxane ratio, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 59

A cicletanine formulation comprising 140 mg of (+) cicletanine and 60 mg of (−) cicletanine is administered via an oral route once daily, to patients with asthma or COPD or pulmonary fibrosis. Cicletanine is administered alone or in combination with other classes of drugs as discussed above.

As a consequence pulmonary function is improved, systemic blood pressure remains within acceptable limits, and in addition one or more of the following parameters is improved: FEV1, O2 saturation, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing midrange fluid overload.

### EXAMPLE 60

A cicletanine formulation comprising 120 mg of (+) cicletanine and 80 mg of (−) cicletanine is administered via an oral route once daily, to patients with asthma or COPD or pulmonary fibrosis. Cicletanine is administered alone or in combination with other classes of drugs as discussed above.

As a consequence pulmonary function is improved, systemic blood pressure remains within acceptable limits, and in addition one or more of the following parameters is improved: FEV1, O2 saturation, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 61

A cicletanine formulation comprising 100 mg of (+) cicletanine and 100 mg of (−) cicletanine is administered via an oral route once daily, to patients with asthma, COPD, or pulmonary fibrosis. Cicletanine is administered alone or in combination with other classes of drugs as discussed above.

As a consequence pulmonary function is improved or stabilized, blood pressure remains within acceptable limits, and in addition one or more of the following parameters is improved: FEV1, O2 saturation, walking distance, prostacyclin/thromboxane ratio, and NYHA functional class score. Metabolic parameters remain unchanged or improved. Such improvements remain above baseline for at least 3 months.

Such a formulation has a mid range diuretic effect and is most appropriate for patients experiencing intermediate fluid overload.

### EXAMPLE 62

A cicletanine formulation comprising 80 mg of (+) cicletanine and 120 mg of (−) cicletanine is administered via an oral route once daily, to patients with asthma or COPD or pulmonary fibrosis. Cicletanine is administered alone or in combination with other classes of drugs as discussed above.

As a consequence pulmonary function is improved, systemic blood pressure remains within acceptable limits, and in addition one or more of the following parameters is improved: FEV1, O2 saturation, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 63

A cicletanine formulation comprising 60 mg of (+) cicletanine and 140 mg of (−) cicletanine is administered via an oral route once daily, to patients with asthma or COPD or pulmonary fibrosis. Cicletanine is administered alone or in combination with other classes of drugs as discussed above.

As a consequence pulmonary function is improved, systemic blood pressure remains within acceptable limits, and in addition one or more of the following parameters is improved: FEV1, O2 saturation, walking distance, prostacyclin/thromboxane ratio, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 64

A cicletanine formulation comprising 40 mg of (+) cicletanine and 160 mg of (−) cicletanine is administered via an oral route once daily, to patients with asthma or COPD or pulmonary fibrosis. Cicletanine is administered alone or in combination with other classes of drugs as discussed above.

As a consequence pulmonary function are improved, systemic blood pressure remains within acceptable limits, and in addition one or more of the following parameters is improved: FEV1, O2 saturation, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effect and is most appropriate for patients experiencing modest fluid overload.

### EXAMPLE 65

A cicletanine formulation comprising 20 mg of (+) cicletanine and 180 mg of (−) cicletanine is administered via an oral route once daily, to patients with asthma or COPD or pulmonary fibrosis. Cicletanine is administered alone or in combination with other classes of drugs as discussed above.

As a consequence pulmonary function is improved, systemic blood pressure remains within acceptable limits, and in addition one or more of the following parameters improved: FEV1, O2 saturation, walking distance, prostacyclin/thromboxane ratio, and NYHA functional class score. Metabolic parameters remain unchanged or improved. Such improvements remain above baseline for at least 3 months.

Such a formulation has a mild diuretic effect and strong nitric oxide effects and is most appropriate for patients experiencing modest fluid overload or no fluid overload.

### EXAMPLE 66

A cicletanine formulation comprising 200 mg of (−) cicletanine is administered via an oral route once daily, to patients with asthma or COPD or pulmonary fibrosis. Cicletanine is administered alone or in combination with other classes of drugs as discussed above.

As a consequence pulmonary function is improved, systemic blood pressure remains within acceptable limits, and in addition one or more of the following parameters is improved: FEV1, O2 saturation, walking distance, prostacyclin/thromboxane ratio, and NYHA functional class score. Metabolic parameters remain unchanged or improved. Such improvements remain above baseline for at least 3 months.

This formulation has little or no diuretic effect, and maximal nitric oxide effect, and is best suited to patients with no fluid overload.

### EXAMPLE 67

A cicletanine formulation comprising 200 mg of (+) cicletanine is administered via an oral route once daily, to patients with heart failure or cor pulmonale. Cicletanine is administered alone or in combination with other classes of drugs as discussed above.

As a consequence cardiac function is improved or stabilized, systemic blood pressure remains within acceptable limits, and in addition one or more of the following parameters is improved: Natriuretic peptide levels, cardiac output, walking distance, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 68

A cicletanine formulation comprising 180 mg of (+) cicletanine and 20 mg of (−) cicletanine is administered via an oral route once daily, to patients with heart failure or cor pulmonale. Cicletanine is administered alone or in combination with other classes of drugs as discussed above.

As a consequence function is improved, systemic blood pressure remains within acceptable limits, and in addition one or more of the following parameters is improved: Natriuretic peptide levels, cardiac output, walking distance, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 69

A cicletanine formulation comprising 160 mg of (+) cicletanine and 40 mg of (−) cicletanine is administered via an oral route once daily, to patients with heart failure or cor pulmonale. Cicletanine is administered alone or in combination with other classes of drugs as discussed above.

As a consequence cardiac function is improved, systemic blood pressure remains within acceptable limits, and in addition one or more of the following parameters is improved: Natriuretic peptide levels, cardiac output, walking distance, and NYHA functional class score. Metabolic parameters remain unchanged or improved. Such improvements remain above baseline for at least 3 months.

Such a formulation has a prominent diuretic effect and is most appropriate for patients experiencing substantial fluid overload.

### EXAMPLE 70

A cicletanine formulation comprising 140 mg of (+) cicletanine and 60 mg of (−) cicletanine is administered via an oral route once daily, to patients with heart failure or cor pulmonary. Cicletanine is administered alone or in combination with other classes of drugs as discussed above.

As a consequence cardiac function is improved, systemic blood pressure remains within acceptable limits, and in addition one or more of the following parameters is improved: Natriuretic peptide levels, cardiac output, walking distance, and NYHA functional class score. Metabolic parameters remain unchanged or improved. Such improvements remain above baseline for at least 3 months.

Such a formulation has a less prominent diuretic effect and is most appropriate for patients experiencing midrange fluid overload.

### EXAMPLE 71

A cicletanine formulation comprising 120 mg of (+) cicletanine and 80 mg of (−) cicletanine is administered via an oral route once daily, to patients with heart failure or cor pulmonale. Cicletanine is administered alone or in combination with other classes of drugs as discussed above.

As a consequence cardiac function is improved, systemic blood pressure remains within acceptable limits, and in addition one or more of the following parameters is improved: Natriuretic peptide levels, cardiac output, walking distance, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 72

A cicletanine formulation comprising 100 mg of (+) cicletanine and 100 mg of (−) cicletanine is administered via an oral route once daily, to patients with heart failure or cor pulmonale. Cicletanine is administered alone or in combination with other classes of drugs as discussed above.

As a consequence cardiac function is improved or stabilized, blood pressure remains within acceptable limits, and in addition one or more of the following parameters is improved: Natriuretic peptide levels, cardiac output, walking distance, and NYHA functional class score. Metabolic parameters remain unchanged or improved. Such improvements remain above baseline for at least 3 months.

Such a formulation has a mid range diuretic effect and is most appropriate for patients experiencing intermediate fluid overload.

### EXAMPLE 73

A cicletanine formulation comprising 80 mg of (+) cicletanine and 120 mg of (−) cicletanine is administered via an oral route once daily, to patients with heart failure or cor pulmonale. Cicletanine is administered alone or in combination with other classes of drugs as discussed above.

As a consequence cardiac function is improved, systemic blood pressure remains within acceptable limits, and in addition one or more of the following parameters is improved: Natriuretic peptide levels, cardiac output, walking distance, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 74

A cicletanine formulation comprising 60 mg of (+) cicletanine and 140 mg of (−) cicletanine is administered via an oral route once daily, to patients with heart failure or cor pulmonale. Cicletanine is administered alone or in combination with other classes of drugs as discussed above.

As a consequence cardiac function is improved, systemic blood pressure remains within acceptable limits, and in addition one or more of the following parameters is improved: Natriuretic peptide levels, cardiac output, walking distance, and NYHA functional class score. Metabolic parameters remain unchanged or improved. Such improvements remain above baseline for at least 3 months.

Such a formulation has a less prominent diuretic effect and is most appropriate for patients experiencing intermediate fluid overload.

### EXAMPLE 75

A cicletanine formulation comprising 40 mg of (+) cicletanine and 160 mg of (−) cicletanine is administered via an oral route once daily, to patients with heart failure or cor pulmonale. Cicletanine is administered alone or in combination with other classes of drugs as discussed above.

As a consequence cardiac function is improved, systemic blood pressure remains within acceptable limits, and in addition one or more of the following parameters is improved: Natriuretic peptide levels, cardiac output, walking distance, and NYHA functional class score. Metabolic parameters remain unchanged or improved. Such improvements remain above baseline for at least 3 months.

Such a formulation has a mild diuretic effect and strong nitric oxide effect and is most appropriate for patients experiencing modest fluid overload.

### EXAMPLE 76

A cicletanine formulation comprising 20 mg of (+) cicletanine and 180 mg of (−) cicletanine is administered via an oral route once daily, to patients with heart failure or cor pulmonale. Cicletanine is administered alone or in combination with other classes of drugs as discussed above.

As a consequence cardiac function is improved, systemic blood pressure remains within acceptable limits, and in addition one or more of the following parameters is improved: Natriuretic peptide levels, cardiac output, walking distance, and NYHA functional class score. Metabolic parameters remain unchanged or improved. Such improvements remain above baseline for at least 3 months.

Such a formulation has a mild diuretic effect and strong nitric oxide effects and is most appropriate for patients experiencing modest fluid overload or no fluid overload.

### EXAMPLE 77

A cicletanine formulation comprising 200 mg of (−) cicletanine is administered via an oral route once daily, to patients with heart failure or cor pulmonale. Cicletanine is administered alone or in combination with other classes of drugs as discussed above.

As a consequence cardiac function is improved, systemic blood pressure remains within acceptable limits, and in addition one or more of the following parameters is improved: Natriuretic peptide levels, cardiac output, walking distance, and NYHA functional class score. Metabolic parameters remain unchanged or improved. Such improvements remain above baseline for at least 3 months.

This formulation has little or no diuretic effect, and maximal nitric oxide effect, and is best suited to patients with no fluid overload.

### EXAMPLE 78

A non-racemic combination drug is formulated into a pill, capsule or other dosage form of mixed composition comprising approximately 90 mg of the (+) enantiomer of Cicletanine and is combined with 10 mg of the (−) enantiomer of Cicletanine and is administered orally, once a day, to subjects suffering from pulmonary hypertension. The nonracemic formulated drug is administered, alone or in combination with drugs from other classes, either as a first-line drug or as a drug given in addition to or as a replacement for a previous/current drug given for pulmonary hypertension.

When this non-racemic formulation is administered to appropriate subjects (including, but not limited to, those suggested above) pulmonary blood pressure favorably falls and a positive effect upon metabolic parameters (in particular, blood glucose levels, glucose tolerance, blood triglyceride levels, blood cholesterol [total, LDL and Ha] levels) will either be positive or neutral, as compared to controls.

These results indicate that the non-racemic drug formulation above has a predominantly-diuretic effect, while having some pulmonary vasorelaxant and organ-protective effects. Additionally, the potassium-lowering effect and the effect of this drug upon lipids and cholesterol is healthier and less pronounced than that of the thiazide-type diuretics.

### EXAMPLE 79

A non-racemic combination drug is formulated into a pill, capsule or other dosage form of mixed composition of approximately 80 mg of the (+) enantiomer of Cicletanine and is combined with 20 mg of the (−) enantiomer of Cicletanine and is administered orally, once a day, to subjects suffering symptoms from one or more of the following descriptions: pulmonary hypertension. The formulated drug is administered either as a first-line drug or as a drug given in addition to, or as a replacement for a previous/current drug given for pulmonary hypertension.

When this non-racemic formulation is administered to appropriate subjects (including but not limited to those suggested above) pulmonary blood pressure falls favorably and effects upon metabolic parameters (in particular, blood glucose levels, glucose tolerance, blood triglyceride levels, blood cholesterol [total, LDL and HDL] levels) are positive or remain neutral to minimal.

These results indicate that the non-racemic drug formulation above has a predominantly-diuretic effect, while having some pulmonary vasorelaxant and organ-protective effects. Additionally, the potassium-lowering effect and the effect of this drug upon lipids and cholesterol are healthier and less pronounced than that of the thiazide-type diuretics.

### EXAMPLE 80

A non-racemic combination drug is formulated into a pill, capsule or other dosage form of mixed composition of approximately 70 mg of the (+) enantiomer of Cicletanine combined with 30 mg of the (−) enantiomer of Cicletanine and is administered orally once a day to subjects suffering symptoms from one or more of the following descriptions: pulmonary hypertension, either alone or combined with drugs from other classes or pulmonary hypertension in the presence of mildly or moderately-elevated triglycerides, cholesterol or blood glucose, but not in the presence of actual metabolic syndrome. The nonracemic formulated drug is administered, alone or in combination with drugs from other classes, either as a first-line drug or as a drug given in addition to or as a replacement for a previous/current drug given for pulmonary hypertension.

When this non-racemic formulation is administered to appropriate subjects (including, but not limited to those suggested above) pulmonary blood pressure falls favorably, and effects upon metabolic parameters (in particular, blood glucose levels, glucose tolerance, blood triglyceride levels, blood cholesterol [total, LDL and HDL] levels) are positive or remain neutral to minimal.

These results indicate that the non-racemic drug formulation above has a predominantly-diuretic effect, while having some vasorelaxant and organ-protective effects. The diuretic effects, however, will be more pronounced than the others.

Additionally, the potassium-lowering effect and the effect of this drug upon lipids and cholesterol are healthier and less pronounced than that of the thiazide-type diuretics.

### EXAMPLE 81

A non-racemic combination drug is formulated into a pill, capsule or other dosage form of mixed composition of approximately 60 mg of the (+) enantiomer of Cicletanine combined with 40 mg of the (−) enantiomer of Cicletanine and is administered orally, once a day, to subjects suffering symptoms from one or more of the following descriptions: pulmonary hypertension in the presence of mildly or moderately-elevated triglycerides, cholesterol, or blood glucose both in and out of the presence of actual metabolic syndrome. The drug is administered either as a first-line drug or as a drug given in addition to, or as a replacement for a previous/current drug given for pulmonary hypertension.

When this non-racemic formulation is administered to appropriate subjects (including, but not limited to those suggested above) pulmonary blood pressure falls favorably and effects upon metabolic parameters (in particular, blood glucose levels, glucose tolerance, blood triglyceride levels, blood cholesterol [total, LDL and HDL] levels) are positive or remain neutral to minimal.

These results indicate that the non-racemic drug formulation above have a markedly-diuretic effect, as well as pulmonary vasorelaxant and organ-protective effects. The diuretic effects, however, may be more pronounced than the other effects. Additionally, the potassium-lowering effect and the effect of this drug upon lipids and cholesterol will be healthier and less pronounced than that of the thiazide-type diuretics.

### EXAMPLE 82

A non-racemic combination drug is formulated into a pill, capsule or other dosage form of mixed composition of approximately 40 mg of the (+) enantiomer of Cicletanine combined with 60 mg of the (−) enantiomer of Cicletanine and is administered orally, once a day, to subjects suffering symptoms from one or more of the following descriptions: uncomplicated pulmonary hypertension, either alone or combined with drugs from other classes; hypertension in the presence of mildly or moderately-elevated triglycerides, cholesterol or blood glucose; pulmonary hypertension in the presence of diabetes or metabolic syndrome. The drug is administered either as a first-line drug or as a drug given in addition to, or as a replacement for a previous/current drug given for pulmonary hypertension or other pulmonary diseases or complications thereof.

When this non-racemic formulation is administered to appropriate subjects (including, but not limited to those suggested above) pulmonary blood pressure falls favorably, and effects upon metabolic parameters (in particular, blood glucose levels, glucose tolerance, blood triglyceride levels, blood cholesterol [total, LDL and HDL] levels) are positive or remain neutral to minimal.

These results indicate that the non-racemic drug formulation above has a diuretic effect, as well as pulmonary vasorelaxant and organ-protective effects. The pulmonary vasorelaxant effects, however, are more pronounced than the other effects. Additionally, the potassium-lowering effect and the effect of this drug upon lipids and cholesterol is healthier and less pronounced than that of the thiazide-type diuretics.

### EXAMPLE 83

A non-racemic combination drug is formulated into a pill, capsule or other dosage form of mixed composition of approximately 30 mg of the (+) enantiomer of Cicletanine combined with 70 mg of the (−) enantiomer of Cicletanine and is administered orally, once a day, to subjects suffering symptoms from one or more of the following descriptions: uncomplicated pulmonary hypertension, either alone or combined with drugs from other classes; hypertension in the presence of mildly or moderately-elevated triglycerides, cholesterol or blood glucose; pulmonary hypertension in the presence of diabetes or metabolic syndrome. The drug is administered either as a first-line drug or as a drug given in addition to, or as a replacement for a previous/current drug given for pulmonary hypertension, other pulmonary diseases, or complications thereof.

When this non-racemic formulation is administered to appropriate subjects (including, but not limited to those suggested above) pulmonary blood pressure falls favorably and effects upon metabolic parameters (in particular, blood glucose levels, glucose tolerance, blood triglyceride levels, blood cholesterol [total, LDL and HDL] levels) are positive or remain neutral to minimal.

These results indicate that the non-racemic drug formulation above has a diuretic effect, as well as pulmonary vasorelaxant and organ-protective effects. The vasorelaxant and organ protective effects, however, are more pronounced than the other effects. Additionally, the potassium-lowering effect and the effect of this drug upon lipids and cholesterol is healthier and less pronounced than that of the thiazide-type diuretics.

### EXAMPLE 84

A non-racemic combination drug is formulated into a pill, capsule or other dosage form of mixed composition of approximately 20 mg of the (+) enantiomer of Cicletanine combined with. 80 mg of the (−) enantiomer of Cicletanine and is administered orally, once a day, to subjects suffering symptoms from one or more of the following descriptions: uncomplicated pulmonary hypertension, either alone or combined with drugs from other classes; hypertension in the presence of mildly or moderately-elevated triglycerides, cholesterol or blood glucose. The drug is administered either as a first-line drug or as a drug given in addition to, or as a replacement for a previous/current drug given for pulmonary hypertension, other pulmonary diseases, or complications thereof.

When this non-racemic formulation is administered to appropriate subjects (including, but not limited to those suggested above) pulmonary blood pressure falls favorably and effects upon metabolic parameters (in particular, blood glucose levels, glucose tolerance, blood triglyceride levels, blood cholesterol [total, LDL and HDL] levels) are positive or remain neutral to minimal.

These results indicate that the non-racemic drug formulation above has a diuretic effect, as well as vasorelaxant and organ-protective effects. The vasorelaxant and organ protective effects, however, are more pronounced than the diuretic effect. Additionally, the potassium-lowering effect and the effect of this drug upon lipids and cholesterol are healthier and less pronounced than that of the thiazide-type diuretics.

### EXAMPLE 85

A non-racemic combination drug is formulated into a pill, capsule or other dosage form of mixed composition of approximately 10 mg of the (+) enantiomer of Cicletanine combined with 90 mg of the (−) enantiomer of Cicletanine and is administered orally, once a day, to subjects suffering symptoms from one or more of the following descriptions: pulmonary hypertension, either alone or combined with drugs from other classes; pulmonary hypertension in the presence of mildly or moderately-elevated triglycerides, cholesterol or blood glucose; pulmonary hypertension in the presence of diabetes or metabolic syndrome. The drug is administered either as a first-line drug or as a drug given in addition to, or as a replacement for a previous/current drug given for pulmonary hypertension, other pulmonary diseases, or complications thereof.

When this non-racemic formulation is administered to appropriate subjects (including, but not limited to those suggested above) pulmonary blood pressure falls favorably and effects upon metabolic parameters (in particular, blood glucose levels, glucose tolerance, blood triglyceride levels, blood cholesterol [total, LDL and HDL] levels) are positive or remain neutral to minimal.

These results indicate that the non-racemic drug formulation above has a diuretic effect, as well as pulmonary vasorelaxant and organ-protective effects. The pulmonary vasorelaxant and organ protective effects, however, are more pronounced than the diuretic effect. Additionally, the potassium-lowering effect and the effect of this drug upon lipids and cholesterol are healthier and less pronounced than that of the thiazide-type diuretics.

In some embodiments of the invention, the various cicletanine compositions may be the sole therapeutic agent of the formulation, in other embodiments, a second agent or agents may be included along with the cicletanine composition. “Second agent”, as used herein, refers to any agent other than the cicletanine compositions; “second agent”, as such is a generic term that may include a plurality of agents that are members of this non-cicletanine class. Such second agents may be, by themselves, effective agents for increasing prostacyclin, antagonizing endothelin activity, inhibiting phosphodiesterase activity, inhibiting histaminergic activity, inhibition of calcium-channel activity, enhancing cGMP activity, acting as a Nitrogen donor or NO enhancer, and/or treating any complications associated with pulmonary diseases. In many cases, the range of diseases and associated complications or sequalae that receive therapeutic benefit from formulations consisting only of cicletanine compositions are understood to be the same as those that receive benefit from formulations that include both cicletanine, of varying enantiomeric composition, and a second therapeutic agent.

In accordance with some embodiments of the present invention, an oral formulation is disclosed, comprising a therapeutically effective amount of cicletanine alone or in combination with a second agent such that the overall formulation improves pulmonary hemodynamics without inappropriately lowering systemic blood pressure. Cicletanine might be favorable in this regard because it tends not to lower systemic blood pressure in patients who do not have systemic hypertension. This phenomenon of not lowering systemic blood pressure in systemic normotensives has been observed by the inventors to be more marked than that of other blood-pressure lowering agents. The inventors propose that this is due to cicletanine correcting the root of endogenous deficits (e.g., reversing the decoupling endothelial nitric oxide synthase [eNOS]) rather than over-riding a dysfunctional biochemical cascade by endeavoring to counterbalance downstream phenomena (such downstream counterbalancing is what is done by the major classes of vasorelaxant antihypertensives: ACE inhibitors, angiotensin receptor blockers, calcium-channel blockers and beta blockers).

In another embodiment, a method is disclosed for treating and/or preventing a condition or complication associated with pulmonary diseases such as asthma or other chronic obstructive pulmonary diseases. Cicletanine is thought by the inventors to be of particular interest here, because the inventors have observed that cicletanine, in addition to enhancing systemic prostacyclin and enhancing the coupling of endothelial nitric oxide synthase (eNOS), will also inhibit the excessive histaminergic activity associated with asthma or COPD. This antihistaminergic activity is proposed to be associated with the (−) stereoisomer, but not the (+) stereoisomer of cicletanine.

In another embodiment, the method comprises administering cicletanine (in racemic, non-racemic or pure stereoisomeric forms) via aerosol delivery to the lungs and administering a second pulmonary hypertension agent that acts either as a calcium-channel blocker, phosphodiesterase in inhibitor, prostacyclin analogue or endothelin antagonist, cGMP enhancer, or Nitrogen donor.

In various embodiments of the inventive method, the therapeutically effective amount of the cicletanine is sufficient to mitigate a side effect of the second agent. In another aspect of the method, the amounts of the cicletanine and second agents are sufficient to produce a synergistic antihypertensive effect. In yet another aspect the addition of cicletanine enhances the duration of action of the second agent or reduces the development of tolerance to the second agent. In a further aspect, the use of cicletanine allows for a lower dosage of a second agent treating pulmonary hypertension, thereby decreasing the frequency or severity of the adverse events associated with that second agent.

Embodiments of the present invention may make use of cicletanine as an inducer of prostacyclin, although cicletanine may operate through other mechanisms as well, including diuresis. Cicletanine, in the course of standard synthesis procedures, naturally occurs as a racemic mixture of equal proportions of a positive (+) and a negative (−) enantiomer, however, embodiments of the present invention include formulations that consist purely of either the positive (+) or negative (−) enantiomer, as well as formulations with non-racemic mixtures that may vary in relative proportions, ranging from, for example a formulation with a proportion of about 99% (+) enantiomer: about 1% (−) enantiomer to a formulation with a proportion of about 1% (+) enantiomer: about 99% (−) enantiomer.

In some embodiments of the invention, the combination therapies may comprise fixed doses (of each component), in single-tablet form, single-capsule form, or other combined-dosage forms. In one example, combination of therapies within a single tablet is meant to simplify treatment regimens, and thereby support patient compliance. Further, by way of example, doses of the combined agents relative to each other are fixed, based on supporting an appropriate level of simplicity for treatment regimens. The establishment of doses appropriately that are fixed relative to each other still allows for variation in total dosage. Combination therapy, in general, supports appropriate-level dosing in that it allows the application of doses of individual agents lower than those that elicit the unwanted side effects that may occur at higher dose levels. Further, in the case of combining agents that work toward a broadly defined common benefit but which operate through different mechanisms of action, synergistic therapeutic effects may occur. Synergistic effects, by their nature, are not commonly predictable, based solely on an understanding of the respective mechanisms of the combined individual agents.

A therapeutic embodiment of the present invention comprises a prostacyclin, or more particularly, an agonist or an inducer thereof (particularly via upregulation of nitric oxide) such as a composition of cicletanine, in combination with (1) prostacyclin-analogue agents, such as epoprostenol, iloprost, and treprostinil; (2) endothelin antagonists, such as bosentan; (3) PDE inhibitors such a sildenafil; (4) calcium-channel blockers such as nifedipine, amlodipine and nicardipine; (5) a Nitrogen donor or enhancer, such as SNP, and (6) an inducer or enhancer of cGMP, such as ANP.

The combination may be formulated in accordance with the teachings herein to provide a clinical benefit that goes beyond the beneficial effects produced by either drug alone. Such an enhanced clinical benefit may be related to distinct mechanisms of action and/or a synergistic interaction of the drugs.

Aspects of embodiments of the present invention are applicable in the treatment of PH and related disorders for children and adults.

While a number of embodiments of the invention and variations thereof have been described in detail, other modifications and methods of using the disclosed therapeutic combinations and other information disclosed herein will be apparent to those of skill in the art. Accordingly, it should be understood that various applications, modifications, and substitutions may be made of equivalents without departing from the spirit of the invention or the scope of the claims. Various terms have been used in the description to convey an understanding of the invention. It will be understood that a corresponding description of these various terms applies to common linguistic or grammatical variations or forms of these various terms. It well also be understood that therapeutic agents have been identified by trade names, but that these names are provided as contemporary examples, and the invention is not limited by such literal scope, particularly when agents have been further described in terms of their chemical class and mechanism of action. Although the description is generous in its offering of biochemical theory and interpretation of available data in describing the invention, it should be understood that such theory and interpretation do not bind or limit the claims. Further, it should be understood that the invention is not limited to the embodiments set forth herein for purposes of exemplification, but is to be defined only by a fair reading of the appended claims, including the full range of equivalency to which each element thereof is entitled.

1-85. (canceled)

86. A method of treating pulmonary and cardiac disorders comprising administering to a mammal in need thereof, a therapeutic formulation, comprising nitric oxide modulating agents and/or PKC inhibitors alone or in combination with other agents.

87. The method in claim 86, wherein the nitric oxide modulating agent is a furopyridine compound.

88. The method in claim 87, wherein the nitric oxide modulating furopyridine compound is cicletanine.

89. The method in claim 86, wherein the formulation is selected from an oral time release formulation, injectable formulation, including a depot injectable or a subcutaneously placed extended release device, a transmucosal or transdermal formulation, oral immediate release formulation or inhaled formulation.

90. The method in claim 88, wherein the cicletanine formulation is selected from a racemic mixture of cicletanine isomers, a non-racemic mixture of cicletanine isomers, a pure formulation of the (+) cicletanine isomer, or a pure formulation of the (−) cicletanine isomer.

91. The method in claim 88, wherein the cicletanine formulation is a non-racemic mixture of two enantiomers that will yield blood concentrations of approximately equal amounts of (+) and (−) enantiomers or will optimize diuretic and non-diuretic effects of cicletanine.

92. The method in claim 87, wherein the furopyridine formulation is selected from a racemic mixture of furopyridine isomers, a non-racemic mixture of furopyridine isomers, a pure formulation of the (+) furopyridine isomer or pure formulation of the (−) furopyridine isomer.

93. The method in claim 87, wherein the furopyridine is used to treat pulmonary hypertension in adults or children.

94. The method in claim 93, wherein the pulmonary hypertension treated is selected from WHO Group I (pulmonary arterial hypertension), WHO Group II (pulmonary venous hypertension), WHO Group III (pulmonary hypertension associated with hypoxemia), WHO Group IV (pulmonary hypertension associated with chronic thrombic and/or embolic disease) or WHO Group V (pulmonary hypertension associated with other/miscellaneous disease).

95. The method in claim 94, wherein the pulmonary hypertension treated is WHO Group V (pulmonary hypertension associated with other/miscellaneous disease) selected from sarcoidosis, pulmonary Langerhans-cell histiocytosis, lymphangiomatosis, compression of pulmonary vessels secondary to adenopathy, tumor, or fibrosing mediastinitis.

96. The method in claim 86, wherein the formulation comprises a furopyridine compound and an eNOS cofactor or substrate.

97. The method in claim 94, wherein the furopyridine is cicletanine.

98. The method in claim 93, wherein heart failure is involved.

99. The method in claim 94, wherein the pulmonary hypertension being treated is WHO Group II (pulmonary venous hypertension) and involves left-sided heart failure.

100. The method in claim 86, wherein the pulmonary disease involved can be exacerbated by histaminergic activity.

101. The method in claim 86, wherein the pulmonary disease involved is selected from asthma or COPD having an asthmatic component.

102. The method in claim 86, wherein the disease treated is heart failure.

103. The method in claim 102, where the nitric oxide modulating agent is a furopyridine compound.

104. The method in claim 103, wherein the furopyridine compound is a preparation of cicletanine.

105. The method in claim 104, wherein the cicletanine preparation is selected from a racemic mixture of cicletanine isomers, a non-racemic mixture of cicletanine isomers, a pure formulation of the (+) cicletanine isomer, or a pure formulation of the (−) cicletanine isomer.

106. The method in claim 86, comprising a combination of a PKC inhibitor with a nitric oxide modulating agent.

107. The method in claim 105, wherein the non-racemic mixture of cicletanine isomers comprises a predominance of (−)cicletanine over (+)cicletanine.

108. The method in claim 86, wherein the PKC inhibitor is a PKC-β inhibitor.

109. The method in claim 108, wherein the PKC-β inhibitor is ruboxistaurin.

110. The method in claim 86, comprising a combination of ruboxistaurin with a nitric oxide modulating agent.

111. The method in claim 110, wherein the nitric oxide modulating agent is an eNOS-coupling/activating agent comprising a preparation selected from a racemic mixture of cicletanine isomers, a non-racemic mixture of cicletanine isomers, a pure formulation of the (+) cicletanine isomer, or a pure formulation of the (−) cicletanine isomer.

112. The method in claim 111, wherein the non-racemic mixture of cicletanine isomers has a predominance of (−) cicletanine over (+)cicletanine.

113. The method in claim 94, wherein the pulmonary hypertension treated is WHO Group II (pulmonary venous hypertension) and the cicletanine formulation is selected from a racemic mixture of cicletanine isomers, a non-racemic mixture of cicletanine isomers, a pure formulation of the (+) cicletanine isomer, or a pure formulation of the (−) cicletanine isomer.

114. The method for treating pulmonary, cardiac disease and pulmonary hypertension comprising metabolic and cofactor enhancement strategies by improving therapeutic activity of furopyridines, comprising administering the cicletanine formulation in claim 90.

115. The method in claim 93, wherein the diseases treated is selected from heart failure, cor pulmonale, asthma, COPD, pulmonary fibrosis, or combination thereof.

116. The method of treating pulmonary hypertension in claim 97, comprising administering a therapeutically effective dosage of cicletanine.

117. A therapeutic formulation comprising nitric oxide modulating agents and/or PKC inhibitors alone or in combination with other agents for the treatment of pulmonary and cardiac disorders.

118. The therapeutic formulation in claim 117, wherein the nitric oxide modulating agent is a furopyridine compound.

119. The therapeutic formulation in claim 118, wherein the nitric oxide modulating furopyridine compound is cicletanine.

120. The therapeutic formulation in claim 118, wherein the formulation is selected from an oral time release formulation, an injectable formulation, including a depot injectable or a subcutaneously placed extended release device, a transmucosal or transdermal formulation, an oral immediate release formulation, or an inhaled formulation.

121. The therapeutic formulation in claim 119, wherein the cicletanine formulation is selected from a racemic mixture of cicletanine isomers, a non-racemic mixture of cicletanine isomers, pure formulation of (+) cicletanine isomer or pure formulation of (−) cicletanine isomer.

122. The therapeutic formulation in claim 121, wherein the cicletanine formulation is a non-racemic mixture of two enantiomers that will yield blood concentrations of approximately equal amounts of (+) and (−) enantiomers or will optimize diuretic and non-diuretic effects of cicletanine.

123. The therapeutic formulation in claim 121, wherein the cicletanine formulation is a pure formulation of the (+) cicletanine isomer.

124. The therapeutic formulation in claim 121, wherein the cicletanine formulation is a pure formulation of the (−) cicletanine isomer.

125. The therapeutic formulation in claim 118, wherein the furopyridine formulation is a racemic mixture of furopyridine isomers.

126. The therapeutic formulation in claim 125, wherein the furopyridine formulation is selected from a racemic mixture of furopyridine isomers, a non-racemic mixture of furopyridine isomers, a pure formulation of the (+) furopyridine isomer, or a pure formulation of the (−) furopyridine isomer.

127. The therapeutic formulation in claim 117, wherein the furopyridine is used to treat pulmonary hypertension in adults or children.

128. The therapeutic formulation in claim 127, wherein the pulmonary hypertension is selected from WHO Group I (pulmonary arterial hypertension), WHO Group II (pulmonary venous hypertension), WHO Group III (pulmonary hypertension associated with hypoxemia), WHO Group IV (pulmonary hypertension associated with chronic thrombic and/or embolic disease) or WHO Group V (pulmonary hypertension associated with other/miscellaneous disease).

129. The therapeutic formulation in claim 128, wherein the pulmonary hypertension is WHO Group V (pulmonary hypertension associated with other/miscellaneous disease) selected from sarcoidosis, pulmonary Langerhans'-cell histiocytosis, lymphangiomatosis, compression of pulmonary vessels secondary to adenopathy, tumor, or fibrosing mediastinitis.

130. The therapeutic formulation in claim 117, comprising a combination of ruboxistaurin with a nitric oxide modulating agent.

131. The therapeutic formulation in claim 130, wherein the nitric oxide modulating agent is an eNOS-coupling/activating agent comprising a preparation selected from a racemic mixture of furopyridine isomers, a non-racemic mixture of furopyridine isomers, a pure formulation of the (+) furopyridine isomer, or a pure formulation of the (−) furopyridine isomer.

132. The therapeutic formulation in claim 117, wherein the nitric oxide modulating agent is in combination with an agent selected from beta blockers, digitalis derivatives, phosphodiesterase inhibitors, vasodilators or diuretics.

133. The therapeutic formulation in claim 117 wherein the nitric oxide modulating agent is in combination with an agent selected from prostacyclin and analogues thereof, xprostacyclin inducers, endothelin antagonist, phosphodiesterase inhibitors, antihistamine, calcium channel blockers, cGMP activators, or nitrogen or nitric oxide enhancers or donors.

134. The therapeutic formulation in claim 117 wherein the nitric oxide modulating agent is in combination with an agent selected from immune modulators, leukotriene inhibitors, beta agonists, or mast cell inhibitors.

135. The therapeutic formulation in claim 117 wherein the nitric oxide modulating agent is in combination with an agent selected from gemfibrozil, tetrahydrobiopterin, arginine or arginine containing compounds, nitroglyceride, isosorbide dinitrite or combinations thereof.

136. The therapeutic formulation in claim 128, wherein the pulmonary hypertension treated is WHO Group II (pulmonary venous hypertension), and the cicletanine formulation is selected from a racemic mixture of cicletanine isomers, a non-racemic mixture of cicletanine isomers, pure formulation of (+) cicletanine isomer or pure formulation of (−) cicletanine isomer.

137. The therapeutic formulation in claim 121, wherein the non-racemic mixture of cicletanine isomers comprises a predominance of (−) cicletanine over (+) cicletanine.

138. The therapeutic formulation in claim 117, wherein the PKC inhibitor is PKC-β inhibitor.

139. The therapeutic formulation in claim 138 wherein the PKC-β inhibitor is ruboxistaurin.
